(19) United States
(12) Patent Application Publication
(10) Pub. No.: US 2010/0275943 A1
(43) Pub. Date: Nov. 4, 2010

(54) FORMULATIONS FOR MAINTAINING THE FORM OF A SPECIFIC HAIRCUT

(76) Inventors: Robert SALEM, New York, NY (US); Maria Laguardia, Briarcliff Manor, NY (US); Joseph Cincotta, Trumbull, CT (US)

Correspondence Address: Pearl Cohen Zedek Latzer, LLP, 1500 Broadway, 12th Floor, New York, NY, 10036, US

(21) Appl. No.: 12/773,694

(22) Filed: May 4, 2010

Related U.S. Application Data
(60) Provisional application No. 61/175,133, filed on May 4, 2009.

(51) Int. Cl. A45D 7/00; A61K 8/92; A61Q 5/06; A45D 44/00

(52) U.S. Cl. 132/200; 424/70.12; 132/221

(57) ABSTRACT

The invention provides for a dual-formula catalyst composition for maintaining a haircut. The composition comprises: a first formula, and a second formula, wherein at least one of the first formula or the second formula comprises at least two polymers. Each of the polymers may be selected from the group consisting of cationic polymers, anionic polymers, zwitterionic or bimodal polymers and neutral polymers. A method and kit are also provided.

### CROSS-REFERENCE TO RELATED APPLICATIONS

This application claims the benefit of U.S. Provisional App. No. 61/175,133, filed May 4, 2009, the entirety of which is hereby incorporated by reference.

### BACKGROUND

Due to the different attributes that comprise each particular archetypal haircut (e.g., the “shag”, the “blunt,” the “long layers,” the “bob,” and the “pixie”) conventional hair products are not designed for and often cannot achieve and maintain the entire style. For example, an attractive “shag” haircut requires hair strands to have enhanced texture and dimension and requires that the hair strands be lifted at the root so as to yield layers that have fluid movement. In contrast, a “blunt” haircut requires hair strands to be compressed in order to diminish space between strands so that the strands appear uniform.

Due to the various different characteristics required of hair strands in particular haircuts, the actual hairstyle achieved when using conventional styling products may be significantly different from the desired haircut. Conventional hair products such as gels, mousse and hairsprays are used to address single attributes such as root lift, straightening, smoothing, separation, hold, etc. Such products are intended to hold hair strands in the desired attribute. However, these products are not tailored to address all the attributes of specific haircuts. Furthermore, unless combined and/or layered with additional products that “dirty” the hair and makes the style difficult if not impossible to achieve, these products may damage the strength of the hair strand and scalp.

In order to avoid the disadvantages of conventional hair products, a treatment is needed to safely and effectively address the attributes of specific haircuts so that the haircut accurately matches the desired look and maintains the shape and form of the haircut over time. The present invention addresses at least some of the disadvantages of conventional hair styling products and may help achieve effective styling of a particular haircut by applying a dual-formula catalyst mixture to hair shafts that is specifically tailored to the desired haircut.

### BRIEF DESCRIPTION OF THE FIGURES

FIG. 1A illustrates a “bob” haircut. FIG. 1B illustrates a catalyst formula having cationic polymers/macromolecules and neutral polymer/macromolecules applied to a hair shaft in a typical “bob” haircut.

FIG. 2A illustrates a “long layers” haircut. FIG. 2B illustrates a catalyst formula having cationic polymers/macromolecules and neutral polymers/macromolecules applied to a hair shaft in a “long layers” haircut.

FIG. 3A illustrates a “blunt” haircut. FIG. 3B illustrates a catalyst formula having neutral hydrophobic polymers/macromolecules and poly-cationic polymers applied to a hair fiber in a typical “blunt” haircut.

FIG. 4A illustrates a “shag” haircut. FIG. 4B illustrates a catalyst formula having zwitterionic or bimodal polymer/macromolecules applied to a hair shaft in a typical “shag” haircut.

FIG. 5A illustrates a “pixie” haircut. FIG. 5B illustrates a catalyst formula having a polymer/macromolecules complex including neutral polymers/macromolecules, cationic polymers/macromolecules and zwitterionic or bimodal polymer/macromolecules applied to a hair fiber in a typical “pixie” haircut.

### DETAILED DESCRIPTION

The present invention relates to compositions, methods and kits to achieve and maintain a specific hairstyle. The compositions comprise a catalyst formula which is a dual-formula mixture comprising at least two polymers and/or macromolecules in two portions. The polymers and/or macromolecules may be contained in each of the two portions or in one of the portions with the second portion containing collateral ingredients such as emollients, oils, shine enhancers and excipient. The two portions may be held separate from one another until the catalyst formula is ready to be applied to the hair, at which time the particular functionalities of the polymers and/or macromolecules in the dual-formula catalyst may act on the hair fibers in such a manner as to allow the hair fibers to be effectively styled. When combined, the polymers and/or macromolecules help to achieve and maintain a specific haircut style.

In one embodiment of the invention, a composition comprising the catalyst formula may be formulated for treating hair fibers, strands or shafts so that the hair is preferentially shaped for a specific haircut. The composition is formed by combining a first and second portion, wherein at least one of the first and second portions comprises a catalyst formula of a polymers and/or macromolecules selected from the group consisting of coiled polymers, cationic polymers and/or macromolecules, anionic polymers and/or macromolecules, zwitterionic or bimodal polymers and/or macromolecules and neutral polymer/macromolecules. The composition comprises at least two or more polymers/macromolecules that are added in the composition in an amount such that they form 1% to 15% w/w, preferably 1.5% to 13% w/w of the total solution composition. In addition to the catalyst composition, the formula also comprises collateral ingredients. The compositions of the invention are generally formulated specifically for a particular haircut.

In one embodiment of the invention, a kit for styling hair strands is provided into the desired style for a specific haircut comprising a dual dispenser, which comprises a first chamber, a second chamber, and a separator between the first and second chamber. The first chamber contains a first portion which can be the catalyst formula composition of the invention. The second chamber may contain a second portion, selected from the group consisting of polymers/macromolecules and collateral ingredients. The polymeric/macromolecular composition(s) comprise at least one polymer/macromolecule selected from cationic polymers/macromolecules, anionic polymer/macromolecule, zwitterionic or bimodal polymer/macromolecule and neutral polymers/macromolecules. Cationic and anionic polymers may be separated from one another until the dual-formula catalyst composition is ready to be applied to the hair. The dual-formula catalyst may comprise at least two or more polymers/macromolecules selected from cationic polymers/macromolecules, anionic polymers/macromolecules, zwitterionic or bimodal polymers/macromolecules and neutral polymers/macromolecules.

Alternatively, the first and second portions may be contained in separate containers. The kit may also contain styling apparatus such as a comb or brush and instructions for use.

In one embodiment, the invention provides for a method for maintaining a specific haircut, including dispensing a first portion, comprising the catalyst formula of the invention and dispensing a second portion; mixing the first and second portions to form a dual-formula catalyst and applying the dual-formula catalyst onto the hair to shape the hair into the desired haircut style and repeating the application of the dual-formula catalyst on a daily basis or as desired. The second portion may be a polymeric/macromolecular composition or a collateral ingredient composition and the dual-formula catalyst may comprise at least two or more polymers/macromolecules selected from the group consisting of cationic polymers/macromolecules, anionic polymer/macromolecules, zwitterionic or bimodal polymer/macromolecule and neutral polymers/macromolecules.

In one embodiment of the invention, a method for achieving and/or maintaining the style of a specific haircut may include dispensing a first polymeric/macromolecular composition, dispensing a second portion, mixing the first and second portions to form the dual-formula catalyst, applying the dual-formula catalyst onto the hair, and cutting the hair into the specific hairstyle. The second portion may a polymeric/macromolecular composition or a hair emollient composition. The dual-formula catalyst may comprise at least two or more polymers/macromolecules selected from cationic polymers/macromolecules, anionic polymer/macromolecules, zwitterionic or bimodal polymer/macromolecules and neutral polymers/macromolecules.

The term “polymer” as used herein refers to a large, chainlike molecule made of hundreds or thousands of smaller repeating molecular units (monomers). The term “macromolecule” as used herein refers to a large molecule, with a molecular weight of thousands or millions of Daltons. The macromolecule may be made up of a combination of many smaller, similar molecules and can be can be selected from the group consisting of cationic polymers/macromolecules containing at least one positively (+) charged functionality, anionic polymers/macromolecules containing at least one negatively (−) charged functionality, zwitterionic or bimodal polymers/macromolecules containing at least one positive (+) and at least one negative (−) functionality, and neutral polymers that contain no charge.

As used herein, the term “bimodal polymers/macromolecules” refers to polymers/macromolecules having two polymer chains of opposite charge that intertwine, creating a bipolar structure that contains both positive (+) and negative (−) charges.

In certain embodiments of the invention, the polymer/macromolecules may range in molecular weight from 700 daltons to 1,000,000 daltons. In certain embodiments of the invention, the polymer/macromolecules may range in molecular size from 10-10,000 nm. In certain embodiments of the invention, the polymer/macromolecules include hydrogenated castor oil/sebacic acid copolymer, dimethicone, linoleamidopropyl PG dimonium chloride phosphate dimethicone, hydrolyzed wheat protein, a microcrystalline wax, VP/dimethylaminoethylmethacrylate copolymer, polyquaternium-59, PVM/MA copolymer, polyimide-1, polyquaternium-55, VP/VA copolymer, polyacrylate 15 & polyacrylate-17, polyquaternium-91 & polyacrylate 15, polyacrylate-21 & acrylates/dimethylamino methacrylate copolymer, styrene/acrylates copolymer, oxidized polyethene homopolymers, acrylates/hydroxesters acrylates copolymer and VP/methacrylamide/vinyl imidazole copolymer.

Each of the polymer/macromolecules types listed above may also contain side-chains of carbon or silicone (tails) grafted anywhere along the polymer. These “tails” may be hydrophobic (water repelling or non-polar) or hydrophilic (water attracting or polar). In certain embodiments of the invention, the polymeric/macromolecular portions may contain a mixture of hydrophilic and hydrophobic tails.

The two portions of the dual-formula catalyst composition may be a clear portion and a crème portion. The clear and crème portions may also include suitable collateral ingredients such as emollients, shine agents and oils.

The clear portion may contain anionic and/or cationic and/or zwitterionic/bimodal polymers and/or macromolecules and, in some embodiments of the invention, may contain neutral polymers/macromolecules. In certain embodiments of the invention, the clear portion may contain a rheology modifier which is preferably a hydrophobically modified alkali soluble emulsion.

The crème portion may contain natural oils (abyssinian & Avocado), alkanes (C18-21 alkane), paraffin (C13-16 isoparaffin), a castor oil based copolymer (Hydrogenated Castor oil/sebacic acid copolymer), silicones (cyclohexasiloxane, phanyl trimethicone & dimethicone), a cationic phospholipid (linoleamidopropyl PG dimonium chloride phosphate dimethicone), hydrolyzed wheat protein, a microcrystalline wax, a vinyl pyrrolidone copolymer (VP/dimethylaminoethylmethacrylate copolymer), and a UV filter (polyquaternium-59).

Based on the charges and hydrophobic and hydrophilic properties of the polymers/macromolecules and the negative charges naturally found on the hair surface, the dual-formula catalyst mixture may be applied to hair fiber, enabling the hair strands to be easily shaped into a desired style. The dual-formula catalyst mixture shapes the hair fiber using the ionic and hydrophobic or hydrophilic properties of the polymeric/macromolecular composition, as well as controlling the distances and interactions between individual hair fibers.

The two portions comprising the dual-formula catalyst mixture may be mixed together to form the catalyst mixture just prior to use. Separating the two portions of the catalyst mixture until application of the catalyst mixture to the hair prevents the portions from reacting with one another until applied to the hair. The catalyst mixture may then be applied to the hair to achieve and/or maintain a specific hairstyle as needed.

The particular polymer/macromolecular components of the catalyst mixture may be formulated for styling a particular haircut. The polymers/macromolecules may have a plurality of shapes including a spherical, coil, rectangular, cylindrical, tubular, rod and string shapes. Once applied to the hair, these shapes and structures control longitudinal and horizontal distances between and along hair fibers.

Hair is difficult to cut and style into hairstyles that accurately conform to specific desired styles such as “bob,” long layers, “blunt,” “shag,” and “pixie” haircuts. The use of the dual-formula catalyst mixture may result in effectively achieving and/or maintaining the haircut style that is desired.

In one embodiment, a mixed polymer composition for application to “bob” haircut may contain cationic polymers/macromolecules, spherical polymers, and coiled polymers/macromolecules which have a three-dimensional spherical shape and in certain embodiments the cationic polymers may have hydrophobic side-chains. Examples of such polymers include without limitation VP/dimethylaminoethylmethacrylate copolymer and linoleamidopropyl PG dimonium chloride phosphate dimethicone. The mixed polymer composition may also contain neutral polymers in the shape of tightly wound coils. Examples of such polymers include without limitation styrene/acrylate polymers. In a specific embodiment, the formulation includes water, cetyl alcohol, cyclohexasiloxane, VP/methacrylamide/vinyl imidazole copolymer, hydrogenated castor oil/sebacic acid copolymer, stearyl alcohol, C18-21 alkane, ethylhexyl stearate, butylene glycol, panthenol, abyssinian oil, avocado oil, styrene/acrylates copolymer, cyclopentasiloxane, linoleamidopropyl PG dimonium chloride phosphate, dimethicone, ceteareth 20, dimethicone, acrylates/beheneth-25 methacrylate copolymer, VP/dimethylaminoethylmethacrylate copolymer, phenyl trimethicone, microcrystalline wax, polyquaternium-91, sodium PCA, arginine, aspartic acid, PCA, glycine, alanine, serine, valine, sodium lactate, C13-16 isoparaffin, hydrolyzed wheat protein, oleth-20, triethanolamine, polyacrylate 15, polyquaternium-59, proline, threonine, isoleucine, histidine, phenylalanine, fragrance, DMDM hydantoin, disodium EDTA, and iodopropynyl butylcarbamate.

In another embodiment, a polymer composition for application to the “long layers” haircut includes cationic polymers with hydrophobic tails and oblong coil polymers. The polymers include without limitation polyquaternium-91 & polyacrylate-15, VP/Dimethylaminoethylmethacrylate Copolymer and linoleamidopropyl PG dimonium chloride phosphate dimethicone. The composition also includes neutral polymers in the shape of oblong coils. Such polymers include without limitation oxidized Polyethethylene homopolymer, microcrystalline wax and C18-21 Alkane & C13-16 Isoparaffin. In a specific embodiment, the composition for application to the “long layers” haircut includes. In a specific embodiment, the composition includes water, cetyl alcohol, cyclohexasiloxane, acrylates/hydroxesters acrylates copolymer, hydrogenated castor oil/sebacic acid copolymer, stearyl alcohol, C18-21 alkane, ethylhexyl stearate, butylene glycol, panthenol, abyssinian oil, avocado oil, cyclopentasiloxane, linoleamidopropyl PG, dimonium chloride phosphate, dimethicone, ceteareth-20, dimethicone, acrylates/beheneth-25, methacrylate copolymer, VP/dimethylaminoethylmethacrylate copolymer, phenyl trimethicone, oxidized polyethene homopolymers, microcrystalline wax, polyquaternium-91, sodium PCA, arginine, aspartic acid, PCA, glycine, alanine, serine, valine, sodium lactate, C13-16 isoparaffin, hydrolyzed wheat protein, oleth-20, aminomethylpropanol, polyacrylate 15, polyquaternium-59, proline, threonine, isoleucine, histidine, phenylalanine, triethanolamine, fragrance, DMDM hydantoin, disodium EDTA, and iodopropynyl butylcarbamate.

In another embodiment, a polymer composition for application to the “blunt” haircut includes neutral hydrophobic polymer, straight jacket polymers and cylindrical polymers. Such polymers include without limitation dimethicone, microcrystalline wax and linoleamidopropyl PG dimonium chloride phosphate dimethicone. Other polymers include polycationic cylindrical polymers. Such polymers include without limitation polyquaternium-55, polyquaternium-11 and polyquaternium-7. In a specific embodiment, the composition includes water, cetyl alcohol, cyclohexasiloxane, PVM/MA copolymer, hydrogenated castor oil/sebacic acid copolymer stearyl alcohol, C18-21 alkane, ethylhexyl stearate, butylene glycol, panthenol, abyssinian oil, avocado oil, cyclopentasiloxane, linoleamidopropyl PG dimonium chloride phosphate dimethicone, ceteareth-20, dimethicone, acrylates/beheneth-25 methacrylate, copolymer, polyacrylate-21, VP/dimethylaminoethylmethacrylate copolymer, phenyl trimethicone, microcrystalline wax, polyquaternium-91, sodium PCA, arginine, aspartic acid, PCA, glycine, alanine, serine, valine, sodium lactate, C13-16 isoparaffin, hydrolyzed wheat Protein, and polyquaternium-55, acrylates/dimethylamino methacrylate copolymer, PPG-26 Buteth-26, polyimide-1, dimethiconol, triethanolamine, polyacrylate 15, polyquaternium-59, proline, threonine, isoleucine, histidine, phenylalanine, sodium hydroxide, PEG-40 hydrogenated castor Oil, fragrance, DMDM hydantoin, disodium EDTA, and iodopropynyl butylcarbamate.

In another embodiment, a polymer composition for application to the “shag” haircut includes zwitterionic macromolecules/polymer. These polymers include without limitation polyacrylate-21 & acrylates/dimethylamino methacrylate copolymer and polyquaternium-91 and polyacrylate-15. In a specific embodiment, the composition includes water, cetyl alcohol, cyclohexasiloxane, polyimide-1, hydrogenated castor oil/sebacic acid copolymer, stearyl alcohol, C18-21 alkane, VP/VA copolymer, ethylhexyl stearate, butylene glycol, panthenol, abyssinian oil, hydrolyzed wheat protein, avocado oil, cyclopentasiloxane, linoleamidopropyl PG dimonium chloride phosphate, dimethicone, ceteareth-20, dimethicone, acrylates/beheneth-25 methacrylate copolymer, VP/dimethylaminoethylmethacrylate copolymer, phenyl trimethicone, polyacrylate-21, microcrystalline wax, acrylates/dimethylamino methacrylate copolymer, sodium PCA, arginine, polyquaternium-91, aspartic acid, PCA, glycine, alanine, serine, valine, sodium lactate, C13-16 isoparaffin, oleth-20, triethanolamine, polyacrylate 15, polyquaternium-59, proline, threonine, isoleucine, histidine, phenylalanine, fragrance, DMDM hydantoin, disodium EDTA, and iodopropynyl butylcarbamate

In another embodiment, a polymer composition for application to the “pixie” haircut includes longitudinal string-like polymers. These polymers include without limitation neutral vinyl/pyrrolidone hydrophilic polymers such polyvinylpyrrolidone. Other polymers include cationic polymers with hydrophilic tails including without limitation polyquaternium-55, hydroxethylcellulose and zwitterionic or bimodal polymers, including without limitation polyacrylate-21 and acrylates/simethylamino methacrylate copolymer. In a specific embodiment, the composition includes water, cetyl alcohol, cyclohexasiloxane, polyester-5, hydrogenated castor oil/sebacic acid copolymer, stearyl alcohol, C18-21 alkane, VP/VA copolymer, ethylhexyl stearate, butylene glycol, panthenol, polyacrylate-21, abyssinian oil, hydrolyzed wheat protein, avocado oil, cyclopentasiloxane, linoleamidopropyl PG dimonium chloride phosphate dimethicone, ceteareth 20, dimethicone, acrylates/beheneth-25 methacrylate, copolymer VP/dimethylaminoethylmethacrylate copolymer, phenyl trimethicone, microcrystalline wax, acrylates/dimethylamino methacrylate copolymer, sodium PCA, arginine, polyquaternium-91, dimethiconol, aspartic acid, PCA, glycine, alanine, serine, valine, sodium lactate, C13-16 isoparaffin, oleth-20, triethanolamine, polyacrylate 15, polyquaternium-59, proline, threonine, isoleucine, histidine, phenylalanine, fragrance, DMDM hydantoin, disodium EDTA, and iodopropynyl butylcarbamate.

The compositions, kits, methods and tools of the invention may enable the use of a single composition to quickly and effectively style hair with reduced adverse effects on hair strand strength. Advantages of a dual-formula catalyst mixture can additionally include improved control of hair form and position, and easy application to the hair.

The Examples set forth illustrative embodiments of the invention. Other embodiments may be used in addition or instead. Some embodiments may be practiced without all of the details that are disclosed. While the data shown in the Tables and Figures was obtained using certain components, they can be expanded to encompass a wide range of other components.

### EXAMPLE 1

This Example illustrates a formulation for a “bob” haircut.

As shown in Table 1(a), in certain embodiments, the polymers/macromolecules may comprise a clear portion having the following composition with styrene/acrylates copolymer, polyquaternium-91 and polyacrylate-15 and VP/dimethylaminoethylmethacrylate copolymer acting as the catalyst polymers.

TABLE 1(a)

| Component | Range (w/w) |
|---|---|
| Water | qs |
| Disodium EDTA | 0.05-0.15% |
| Acrylates/Beheneth-25 Methacrylate Copolymer | 1.5-6.0% |
| VP/Methacrylamide/Vinyl Imidazole Copolymer | 5.0-20.0% |
| Oleth-20 | 0.15-0.50% |
| Fragrance | 0.05-0.25% |
| DMDM Hydantoin & Iodopropynyl Butylcarbamate | 0.40-0.60% |
| Triethanolamine | 0.15-0.60% |
| Styrene/Acrylates Copolymer | 0.10-5.0% |
| Polyquaternium-91 & Polyacrylate-15 | 0.10-3.0% |
| VP/Dimethylaminoethylmethacrylate Copolymer | 1.0-5.0% |

As shown in Table 1(b), in certain embodiments, the polymers/macromolecules may comprise a crème portion having the following composition with styrene/acrylates copolymer, linoleamidopropyl PG dimonium chloride phosphate dimethicone and polyquaternium-91 and polyacrylate-15 hydrolyzed wheat protein acting as the catalyst polymers.

TABLE 1(b)

| Component | Range (w/w) |
|---|---|
| Water | qs |
| Disodium EDTA | 0.05-0.15% |
| Butylene Glycol | 0.50-2.0% |
| Panthenol | 0.10-0.25% |
| Cetyl Alcohol | 2.0-5.0% |
| Stearyl Alcohol | 1.0-5.0% |
| Ceteareth-20 | 0.30-0.45% |
| Dimethicone 350 cst | 0.10-3.0% |
| Phenyl Trimethicone | 0.20-2.0% |
| C18-21 Alkane & C13-16 Isoparaffin | 1.0-5.0% |
| Hydrogenated Castor Oil/Sebacic Acid Copolymer | 0.10-3.0% |
| Abyssinian Oil | 0.10-1.0% |
| Avocado Oil | 0.10-1.0% |
| Microcrystalline Wax | 0.10-3.0% |
| Ethylhexyl Stearate | 0.20-5.0% |
| Cyclohexasiloxane & Cyclopentasiloxane | 2.0-10.0% |
| Triethanolamine | 0.05-0.50% |
| Styrene/Acrylates Copolymer | 0.05-5.0% |
| Polyquaternium-59 | 0.05-1.5% |
| VP/Dimethylaminoethylmethacrylate Copolymer | 1.0-5.0% |
| Linoleamidopropyl PG dimonium chloride phosphate | 0.10-1.5% |
| dimethicone | |
| Polyquaternium-91 & Polyacrylate 15 | 0.05-3.0% |
| Hydrolyzed Wheat Protein | 0.01-1.0% |
| DMDM Hydantoin & Iodopropynyl Butylcarbamate | 0.40-0.60% |

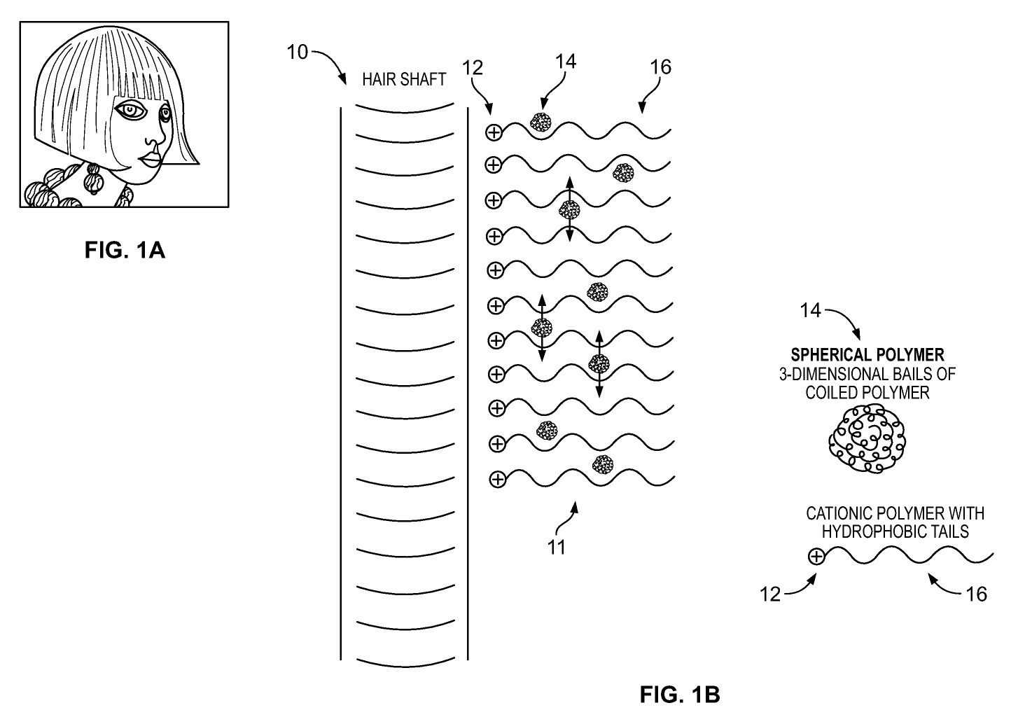

FIG. 1 illustrates a hair shaft 10 of a “bob” haircut. A “bob” haircut is a haircut in which the hairstyle is tilted with a forward momentum. The dual-formula catalyst mixture 11 is shown to comprise cationic polymers/macromolecules 12 with hydrophobic tails 16 to create a base coat on the surface of the hair shaft 10, which is illustrative of all hairs in the cut. The cationic charges at one end of the cationic polymers 12 are attracted to the negative charges naturally found on the hair shaft surface. The hydrophobic tails 16 extend out from the surface of the cationic polymers 12 and create a lubricious and fluid hydrophobic medium which surrounds each hair shaft. This medium imparts a satiny texture and is dry to the touch. Neutral polymers 14 in the shape of tightly wound coils form three-dimensional (3-D) spheres. These 3-D spheres may move freely through the hydrophobic medium. The 3-D spheres may vary in size and molecular weight and provide translational motion at different depths across or along the hair shaft. The force of gravity and physically pushing while blow drying and brushing with a hairbrush may control the motion and shift position of the 3-D spheres in the direction of momentum of the haircut, providing the hair strands with a forward tilt and uniform motion.

### EXAMPLE 2

This Example illustrates a formulation for a “long layers” haircut.

As shown in Table 2(a), in certain embodiments, the polymers/macromolecules may comprise a clear portion having the following composition with polyquaternium-91 and polyacrylate-15 and VP/dimethylaminoethylmethacrylate copolymer acting as the catalyst polymers.

TABLE 2(a)

| Component | Range (w/w) |
|---|---|
| Water | qs |
| Disodium EDTA | 0.05-0.15% |
| Acrylates/Beheneth-25 Methacrylate Copolymer | 1.5-6.0% |
| Acrylates/Hydroxesters Acrylates Copolymer | 4.0-15.0% |
| Oleth-20 | 0.15-0.50% |
| Fragrance | 0.05-0.25% |
| DMDM Hydantoin & Iodopropynyl Butylcarbamate | 0.40-0.60% |
| Aminomethylpropanol | 0.50-1.5% |
| Polyquaternium-91 & Polyacrylate-15 | 0.10-3.0% |
| VP/Dimethylaminoethylmethacrylate Copolymer | 1.0-5.0% |

As shown in Table 2(b), in certain embodiments, the polymers/macromolecules may comprise a crème portion having the following composition with oxidized polyethylene homopolymers, linoleamidopropyl PG dimonium chloride phosphate dimethicone, and polyquaternium-91 and polyacrylate-15 acting as the catalyst polymers.

TABLE 2(b)

| Component | Range (w/w) |
|---|---|
| Water | qs |
| Disodium EDTA | 0.05-0.15% |
| Butylene Glycol | 0.50-2.0% |
| Panthenol | 0.10-0.25% |
| Cetyl Alcohol | 2.0-5.0% |
| Stearyl Alcohol | 1.0-5.0% |
| Ceteareth-20 | 0.30-0.45% |
| Dimethicone 350 cst | 0.10-3.0% |
| Phenyl Trimethicone | 0.20-2.0% |
| C18-21 Alkane & C13-16 Isoparaffin | 1.0-5.0% |
| Hydrogenated Castor Oil/Sebacic Acid Copolymer | 0.10-3.0% |
| Abyssinian Oil | 0.10-1.0% |
| Avocado Oil | 0.10-1.0% |
| Microcrystalline Wax | 0.10-3.0% |
| Ethylhexyl Stearate | 0.20-5.0% |
| Oxidized Polyethene Homopolymers | 0.05-3.0% |
| Cyclohexasiloxane & Cyclopentasiloxane | 2.0-10.0% |
| Triethanolamine | 0.05-0.50% |
| Polyquaternium-59 | 0.05-1.5% |
| VP/Dimethylaminoethylmethacrylate Copolymer | 1.0-5.0% |
| Linoleamidopropyl PG dimonium chloride phosphate | 0.10-1.5% |
| dimethicone | |
| Polyquaternium-91 & Polyacrylate 15 | 0.25-5.0% |
| Hydrolyzed Wheat Protein | 0.01-1.0% |
| DMDM Hydantoin & Iodopropynyl Butylcarbamate | 0.40-0.60% |

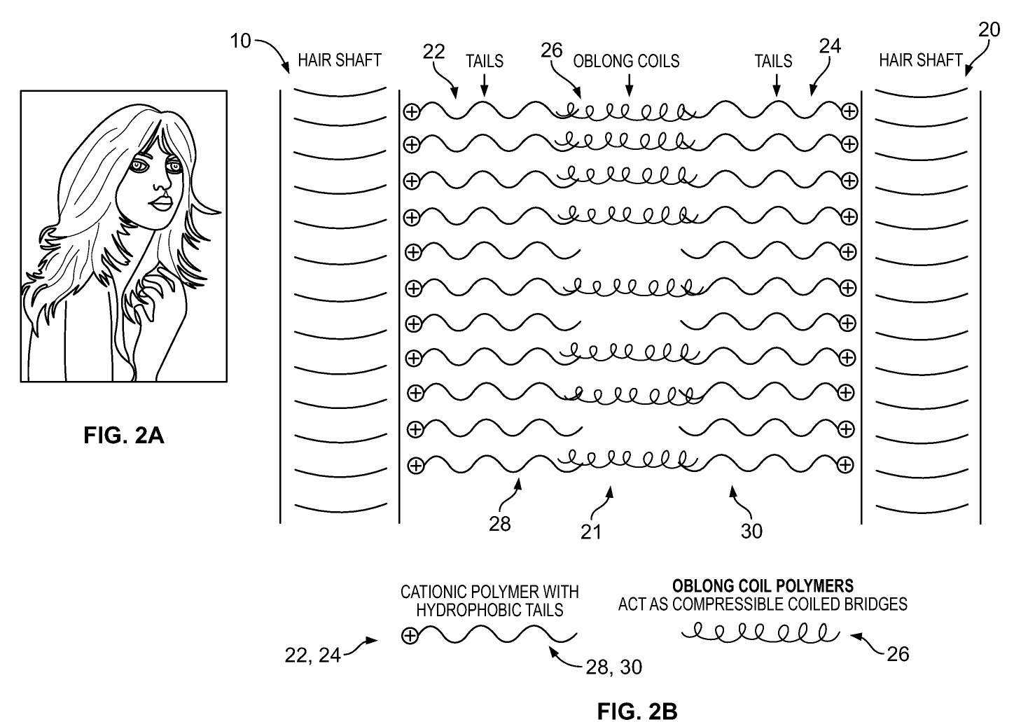

FIG. 2 illustrates a first hair shaft 10 and second hair shaft 20 of a “long layers” haircut. A “long layers” haircut is a haircut in which a platform layer is the longest length of hair and cascading layers of hair are loosely connected to the platform layer creating a flowing movement. The platform layer adds stability and the exterior cascading layers provide movement. The dual-formula catalyst mixture 21 is shown to comprise cationic polymer/macromolecules 22 and 24 with hydrophobic tails 28, and 30 to create a base coat on the surface of each hair shaft 10, 20. The cationic charges on the base coat are attracted to the negative charges naturally found on the hair shaft surface 10, 20. The hydrophobic tails 28 and 30 extend out from the surface of the hair shaft 10, 20 in a coil shaped configuration. A neutral polymer/macromolecule 26 in the shape of an extended oblong coil entangles each of its ends in the coiled tails 28 and 30 of the base coat. This creates a flexible connection between the first and second hair shafts 10, 20 and hair layers. The layers are loosely connected via these coiled bridges. The coiled connections may be extended or compressed.

### EXAMPLE 3

This Example illustrates a formulation for a “blunt” haircut.

As shown in Table 3(a), in certain formulations, the polymers/macromolecules may comprise a clear portion having the following composition with hydroxyethylcellulose, polyvinylpyrrolidone, polyquaternium-11, polyquaternium-7 and polyquaternium-55 acting as the catalyst polymers.

TABLE 3(a)

| Component | Range (w/w) |
|---|---|
| Water | qs |
| Disodium EDTA | 0.05-0.15% |
| Hydroxyethylcellulose | 0.50-1.50% |
| Polyvinylpyrrolidone | 1.0-4.0% |
| Polyquaternium-11 | 1.0-5.0% |
| Polyquaternium-7 | 0.10-0.50% |
| Polyquaternium-55 | 0.20-1.0% |
| Fragrance | 0.05-0.25% |
| DMDM Hydantoin & Iodopropynyl Butylcarbamate | 0.40-0.60% |
| Triethanolamine | 0.10-0.30% |

As shown in Table 3(b), in other formulations, the polymers/macromolecules may comprise a crème portion having the following composition with dimethicone 350 cst, phenyl trimethicone, C18-21 alkane and C13-16 isoparaffin, hydrogenated castor oil/sebacic acid copolymer, Abyssinian oil, avocado oil, microcrystalline wax, ethylhexyl stearate, cyclohexasiloxane and cyclopentasiloxane and dimethiconol acting as the catalyst polymers.

TABLE 3(B)

| Component | Range (w/w) |
|---|---|
| Water | qs |
| Disodium EDTA | 0.05-0.15% |
| Butylene Glycol | 0.50-2.0% |
| Panthenol | 0.10-0.25% |
| Cetyl Alcohol | 2.0-5.0% |
| Stearyl Alcohol | 1.0-5.0% |
| Ceteareth-20 | 0.30-0.45% |
| Dimethicone 350 cst | 0.10-3.0% |
| Phenyl Trimethicone | 0.20-2.0% |
| C18-21 Alkane & C13-16 Isoparaffin | 1.0-5.0% |
| Hydrogenated Castor Oil/Sebacic Acid Copolymer | 0.10-3.0% |
| Abyssinian Oil | 0.10-1.0% |
| Avocado Oil | 0.10-1.0% |
| Microcrystalline Wax | 0.10-3.0% |
| Ethylhexyl Stearate | 0.2-5.0% |
| Cyclohexasiloxane & Cyclopentasiloxane | 0.50-3.0% |
| Cyclopentasiloxane & Dimethiconol | 3.0-8.0% |
| Triethanolamine | 0.05-0.50% |
| Polyquaternium-59 | 0.05-1.5% |
| Polyquaternium-55 | 1.0-10.0% |
| VP/Dimethylaminoethylmethacrylate Copolymer | 1.0-5.0% |
| Linoleamidopropyl PG dimonium chloride phosphate | 0.30-1.5% |
| dimethicone | |
| Polyquaternium-91 & Polyacrylate 15 | 0.05-0.25 |
| Hydrolyzed Wheat Protein | 0.01-1.0% |
| DMDM Hydantoin & Iodopropynyl Butylcarbamate | 0.40-0.60% |

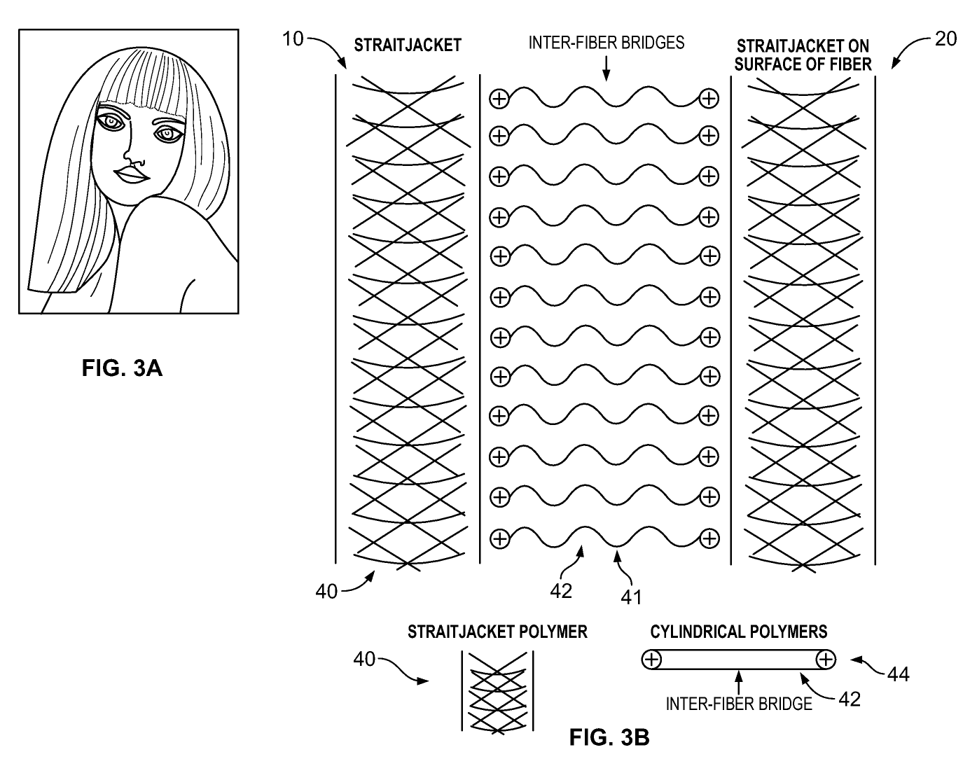

FIG. 3 illustrates a first hair shaft fiber 10 and a second hair shaft fiber 20 of a “blunt” haircut. A “blunt” haircut is a haircut in which all hair shaft fibers are smoothly and tightly aligned to create a straight line. The dual-formula catalyst mixture 41 is shown to comprise neutral hydrophobic polymers/macromolecules 40 that create a crystal clear straitjacket-like matrix around each hair shaft fiber 10, 20 when the polymers/macromolecules are dried. This crystal clear straitjacket accentuates the gloss or shine of the hair. The dried polymer/macromolecular matrix compresses and squeezes the cuticle shaft flat. By flattening the cuticle, the hair shaft fibers may be aligned very closely to one another. Once the cuticle is flattened, inter-fiber bridges 42 are created using poly-cationic cylindrical polymers 44 with short vinyl/pyrrolidone and vinyl/acrylic chains between charged moieties. The compression and cohesiveness of the hair shafts is achieved by flattening the cuticle and forming the inter-fiber bridges.

### EXAMPLE 4

This Example illustrates a formulation for a “shag” haircut.

As shown in Table 4(a), in certain embodiments, the polymers/macromolecules may comprise a clear portion having the following composition with polyacrylate-21 and acrylates/dimethylamino methacrylate copolymer and hydrolyzed wheat protein acting as the catalyst polymers.

TABLE 4(a)

| Component | Range (w/w) |
|---|---|
| Water | qs |
| Disodium EDTA | 0.05-0.15% |
| Acrylates/Beheneth-25 Methacrylate Copolymer | 1.5-6.0% |
| Polyimide-1 | 4.0-15.0% |
| VP/VA Copolymer | 2.0-10.0% |
| Polyacrylate-21 & Acrylates/Dimethylamino Methacrylate | 0.30-5.0% |
| Copolymer | |
| Oleth-20 | 0.15-0.50% |
| Hydrolyzed Wheat Protein | 0.01-1.0% |
| Fragrance | 0.05-0.25% |
| DMDM Hydantoin & Iodopropynyl Butylcarbamate | 0.40-0.60% |
| Triethanolamine | 0.50-1.5% |

As shown in Table 4(b), in certain embodiments, the polymers/macromolecules may comprise a crème portion having the following composition with polyquaternium-91 and polyacrylate-15 and hydrolyzed wheat protein acting as the catalyst polymers.

TABLE 4(b)

| Component | Range (w/w) |
|---|---|
| Water | qs |
| Disodium EDTA | 0.05-0.15% |
| Butylene Glycol | 0.50-2.0% |
| Panthenol | 0.10-0.25% |
| Cetyl Alcohol | 2.0-5.0% |
| Stearyl Alcohol | 1 .0-5.0% |
| Ceteareth-20 | 0.30-0.45% |
| Dimethicone 350 cst | 0.10-3.0% |
| Phenyl Trimethicone | 0.20-2.0% |
| C18-21 Alkane & C13-16 Isoparaffin | 1.0-5.0% |
| Hydrogenated Castor Oil/Sebacic Acid Copolymer | 0.10-3.0% |
| Abyssinian Oil | 0.10-1.0% |
| Avocado Oil | 0.10-1.0% |
| Microcrystalline Wax | 0.10-3.0% |
| Ethylhexyl Stearate | 0.2-5.0% |
| Cyclohexasiloxane & Cyclopentasiloxane | 2.0-10.0% |
| Triethanolamine | 0.05-0.50% |
| Polyquaternium-59 | 0.05-1.5% |
| VP/Dimethylaminoethylmethacrylate Copolymer | 1.0-5.0% |
| Linoleamidopropyl PG dimonium chloride phosphate | 0.50-4.5% |
| dimethicone | |
| Polyquaternium-91 & Polyacrylate 15 | 0.05-0.25% |
| Hydrolyzed Wheat Protein | 0.01-1.0% |
| DMDM Hydantoin & Iodopropynyl Butylcarbamate | 0.40-0.60% |

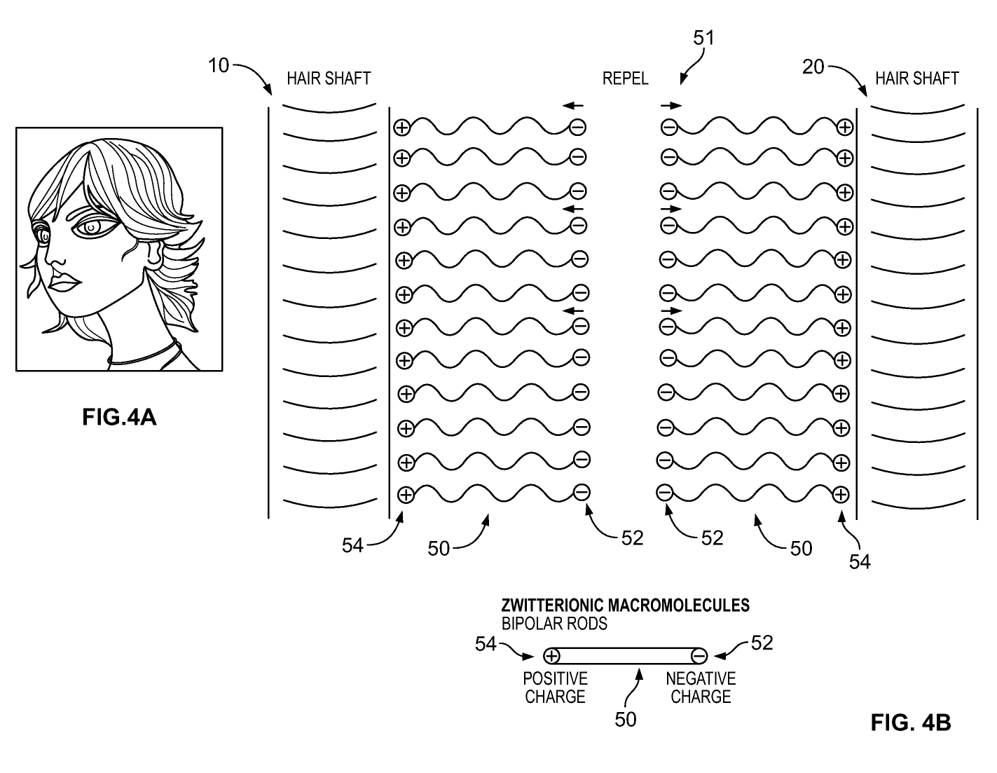

FIG. 4 illustrates a first 10 and second hair 20 shaft of a “shag” haircut. A “shag” haircut is a haircut in which each hair shaft is separated, giving the haircut texture and exhibiting no clumpiness. The haircut has depth and dimension without exhibiting frizz or flyaway. The dual-formula catalyst mixture 51 is shown to comprise base polymers 50 that are zwitterionic macromolecules. The zwitterionic macromolecules are rod-shaped 54 and bipolar. A first end of the zwitterionic macromolecule has a negative charge 52 and the other end has a positive charge 54. A short polyamide chain connects the two oppositely charged ends. The positive end of the macromolecule is attracted to the negative charges naturally found on the hair surface and becomes bound to the hair surface. This leaves the negatively charged end of each macromolecular chain pointing outward. These negative charges pointing outward from one fiber surface repel the negative charges pointing outward from the adjacent hair shaft. This repulsion reduces and prevents clumping or compression of the hair and adds depth and dimension to the hair. The positive charged ends of the zwitterionic macromolecule also eliminate static and flyaway. The dual-formula catalyst mixture provides lift at the root of the hair shaft to yield layers that move.

### EXAMPLE 5

This Example illustrates formulations for a “pixie” haircut.

As shown in Table 5(a), in certain embodiments, the polymers/macromolecules may comprise a clear portion having the following composition with hydroxyethylcellulose, polyacrylate-21 and acrylates/dimethylamino methacrylate copolymer, polyvinylpyrrolidone and polyquaternium-55 acting as the catalyst polymers.

TABLE 5(a)

| Component | Range (w/w) |
|---|---|
| Water | qs |
| Disodium EDTA | 0.05-0.15% |
| Hydroxyethylcellulose | 0.50-1.50% |
| Polyacrylate-21 & Acrylates/Dimethylamino Methacrylate | 0.30-5.0% |
| Copolymer | |
| Polyvinylpyrrolidone | 0.50-1.50% |
| Polyquaternium-55 | 1.0-3.0% |
| Oleth-20 | 0.15-0.50% |
| Fragrance | 0.05-0.25% |
| DMDM Hydantoin & Iodopropynyl Butylcarbamate | 0.40-0.60% |
| Triethanolamine | 0.50-1.5% |

As shown in Table 5(b), in certain embodiments, the polymers/macromolecules may comprise a crème portion having the following composition with VP/dimethylaminoethylmethacrylate copolymer, polyquaternium 55, linoleamidopropyl PG dimonium chloride phosphate dimethicone and polyquaternium-91 and polyacrylate-15 acting as the catalyst polymers.

TABLE 5(b)

| Component | Range (w/w) |
|---|---|
| Water | qs |
| Disodium EDTA | 0.05-0.15% |
| Butylene Glycol | 0.50-2.0% |
| Carbomer | 0.10-0.25% |
| Panthenol | 0.10-0.25% |
| Cetyl Alcohol | 2.0-5.0% |
| Stearyl Alcohol | 1.0-5.0% |
| Ceteareth-20 | 0.30-0.45% |
| Dimethicone 350 cst | 0.10-3.0% |
| Phenyl Trimethicone | 0.20-2.0% |
| C18-21 Alkane & C13-16 Isoparaffin | 1.0-5.0% |
| Hydrogenated Castor Oil/Sebacic Acid Copolymer | 0.10-3.0% |
| Abyssinian Oil | 0.10-1.0% |
| Avocado Oil | 0.10-1.0% |
| Microcrystalline Wax | 0.10-3.0% |
| Ethylhexyl Stearate | 0.20-5.0% |
| Cyclohexasiloxane & Cyclopentasiloxane | 2.0-10.0% |
| Cyclopentasiloxane & Dimethiconol | 0.50-3.0% |
| Triethanolamine | 0.05-0.50% |
| VP/Dimethylaminoethylmethacrylate Copolymer | 1.0-5.0% |
| Polyquaternium-55 | 0.5-5.0% |
| Linoleamidopropyl PG dimonium chloride phosphate | 0.10-3.0% |
| dimethicone | |
| Polyquaternium-91 & Polyacrylate 15 | 0.05-1.0% |
| Hydrolyzed Wheat Protein | 0.01-1.0% |
| DMDM Hydantoin & Iodopropynyl Butylcarbamate | 0.40-0.60% |

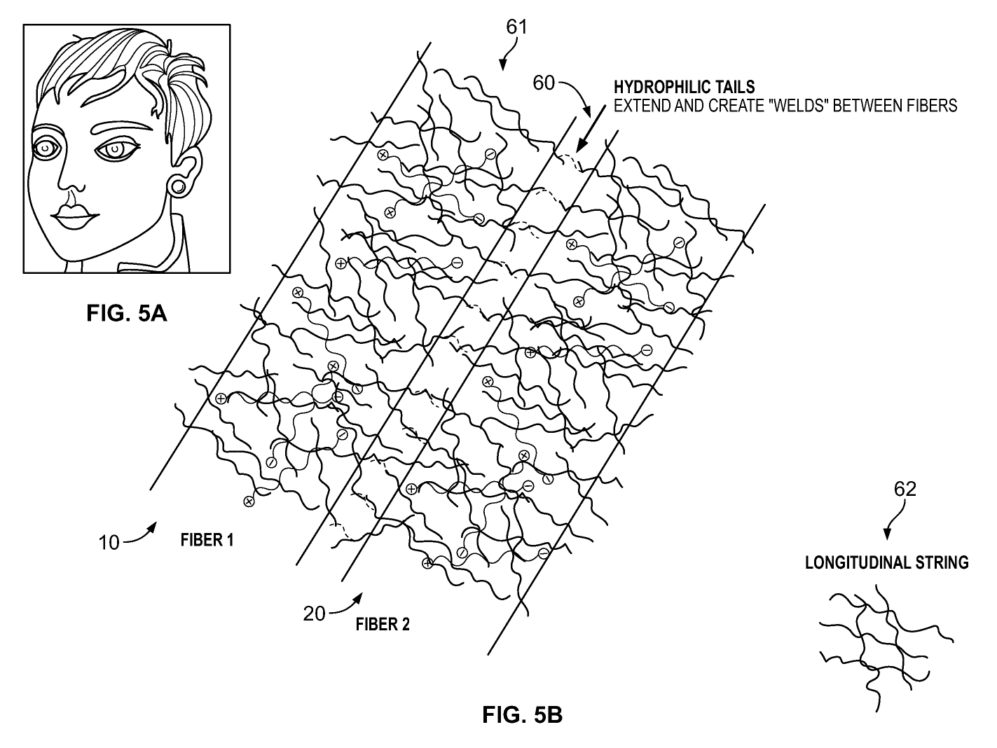

FIG. 5 illustrates a first 10 and second hair fiber 20 of a “pixie” haircut. A “pixie” haircut is a haircut in which hair fibers are contoured to the head and lifted up. The dual-formula catalyst mixture 61 is shown to comprise a polymer/macromolecular complex 60 of several longitudinal string-like polymers 62 intertwined to create a strong and flexible mesh covering each hair shaft in a rigid straight-jacket. Short hydrophilic tails extend above the surface of the hair shaft fibers, which allow inter-fiber welds to be formed when the hair is dry. The mesh or matrix reinforces and strongly holds the hair in the shape, contour or direction in which it is styled. Once the hair dries, the hair is not brittle and does not flake. The longitudinal string-like polymer complex include: neutral vinyl/acrylic hydrophilic polymers, cationic polymers and hydrophilic tails, and zwitterionic or bimodal polymers/macromolecules.

The components, steps, features, objects, benefits and advantages that have been discussed are merely illustrative. None of them, or the discussions relating to them, is intended to limit the scope of protection in any way. Numerous other embodiments are also contemplated, including embodiments that have fewer, additional, and/or different components, steps, features, objects, benefits and advantages. The components and steps may also be arranged and ordered differently.

While certain features of the invention have been illustrated and described herein, many modifications, substitution, changes and equivalents will now occur to those of ordinary skill in the art. It is, therefore, to be understood that the appended claims are intended to cover all such modifications and changes as fall within the spirit of the invention.

We claim:

1. A dual-formula catalyst composition for maintaining a haircut, the composition comprising:
a first formula; and
a second formula,
wherein at least one of the first formula or the second formula comprises at least two polymers, and wherein the polymers are selected from the group consisting of coiled polymers, cationic polymers, anionic polymers, zwitterionic or bimodal polymers and neutral polymers.

2. The composition of claim 1, wherein the weight of the polymers is in the range of about 1% to about 15% based on the total weight of the composition.

3. The composition of claim 1, wherein the first formula is a clear formula and the second formula is a crème formula.

4. The composition of claim 1, wherein the haircut is a bob haircut.

5. The composition of claim 4, wherein each of the polymers is selected from the group consisting of cationic polymers and coiled polymers.

6. The composition of claim 1, wherein the haircut is a long layers haircut.

7. The composition of claim 6, wherein each of the polymers is selected from the group consisting of cationic polymers and neutral polymers in the shape of oblong coils.

8. The composition of claim 1, wherein the haircut is a blunt haircut.

9. The composition of claim 8, wherein each of the polymers is selected from the group consisting of neutral hydrophobic polymers and polycationic cylindrical polymers.

10. The composition of claim 1, wherein the haircut is a shag haircut.

11. The composition of claim 10, wherein each of the polymers is a zwitterionic polymer.

12. The composition of claim 1, wherein the haircut is a pixie haircut.

13. The composition of claim 12, wherein each of the polymers is selected from the group consisting of longitudinal string-like polymers and cationic polymers with hydrophilic tails.

14. A method comprising:
dispensing a clear formula comprising two or more polymers;
dispensing a crème formula comprising two or more polymers;
mixing the clear formula and the crème formula to form a dual-formula catalyst; and
applying the dual-formula catalyst to hair.

15. The method of claim 14, further comprising cutting the hair into a hair cut for which the dual-formula catalyst is suited.

16. The method of claim 14, wherein each of the polymers is selected from the group consisting of coiled polymers, cationic polymers, anionic polymers, zwitterionic or bimodal polymers and neutral polymers.

17. The method of claim 14, wherein the weight of the polymers is in the range of about 1% to about 15% based on the total weight of the composition.

18. A kit comprising:
a first chamber for dispensing a clear formula comprising two or more polymers;
a second chamber for dispensing a crème formula comprising two or more polymers; and
a separator between the first chamber and the second chamber to separate the clear formula from the crème formula until the clear formula and the crème formula are mixed to form a dual-formula catalyst.

19. The kit of claim 18, further comprising a styling apparatus.

20. The kit of claim 18, wherein each of the polymers is selected from the group consisting of coiled polymers, cationic polymers, anionic polymers, zwitterionic or bimodal polymers, and neutral polymers.
